(19) United States
(12) Patent Application Publication
(10) Pub. No.: US 2026/0053553 A1
(43) Pub. Date: Feb. 26, 2026

(54) IRRIGATION WORKFLOW FOR PULSED FIELD ABLATION

(71) Applicant: Biosense Webster (Israel) Ltd., Yokneam (IL)

(72) Inventors: Christopher Thomas Beeckler, Brea, CA (US); Assaf Govari, Haifa (IL); Andres Claudio Altmann, Haifa (IL); Athanassios Papaioannou, Los Angeles, CA (US)

(21) Appl. No.: 19/245,546

(22) Filed: Jun. 23, 2025

Related U.S. Application Data
(60) Provisional application No. 63/686,193, filed on Aug. 23, 2024.

(51) Int. Cl. A61B 18/14; A61B 18/00; A61M 3/02

(52) U.S. Cl. CPC A61B 18/1492; A61M 3/0202; A61B 2018/00267; A61B 2018/00357; A61B 2018/00577; A61B 2018/00642; A61B 2018/00702; A61B 2018/00761; A61B 2018/00839; A61B 2018/0094; A61B 2018/1467; A61B 2218/002

(57) ABSTRACT

A method for irrigation during pulsed field ablation (PFA) includes operating a pump for delivering irrigation in idle mode, receiving a first user command to deliver a PFA sequence and toggling the pump to active mode. The first PFA sequence is delivered after a pre-defined ramp-up delay, and a timer is preset to be ON for a defined working duration. A second user command to delivery a PFA sequence is received and delivered with no imposed delay while the timer is ON. The timer is reset based on the second user command. The pump is toggled to idle mode after the defined working duration lapses.

### CROSS-REFERENCE TO RELATED APPLICATIONS

This application is related to U.S. Provisional Patent Application 63/686,193, filed Aug. 23, 2024, whose disclosure is incorporated herein by reference.

### TECHNICAL FIELD

The present disclosure relates generally to pulsed field ablation for cardiac ablation, and more particularly to an irrigation workflow during delivery of pulsed field energy.

### BACKGROUND

Arrhythmias are abnormal heart rhythms that are typically caused by a small area of cardiac tissue that produces irregular heartbeats. Cardiac ablation is a medical procedure that can be performed to treat an arrhythmia by destroying the area of the cardiac tissue causing the irregular heartbeats. Some medical systems use Pulsed Field Ablation (PFA) to ablate cardiac tissue. PFA is a nonthermal ablation method based on the unrecoverable permeabilization of cell membranes. The permeabilization is caused by short pulses of high voltage delivered to the tissue. Although PFA is not based on heating, there is still may be some local heating during the ablation process. Irrigation is typically applied to regulate the local heating during the ablation process.

### BRIEF DESCRIPTION OF THE DRAWINGS

The present disclosure will be more fully understood from the following detailed description of examples thereof, taken together with the drawings, where like numerals or characters indicate corresponding or like components. Dimensions of components and features shown in the figures are chosen for convenience and clarity of presentation and are not necessarily shown to scale. In the drawings:

FIG. 1 is a simplified block diagram of a catheter-based electrophysiology (EP) mapping and ablation system in accordance with an example of the present disclosure;

FIGS. 2A and 2B are two example timelines for activating irrigation during an electrophysiology procedure that includes Pulsed Field Ablation (PFA) in accordance with an example of the present disclosure;

FIG. 3 is a simplified flow chart of an example method for controlling flowrate of irrigation during an electrophysiology procedure that includes PFA in accordance with an example of the present disclosure; and

FIG. 4 is an example temperature elevation graph showing temperature elevations for two different PFA ablation signals applied for forming a lesion at a selected site in accordance with an example of the present disclosure.

### DETAILED DESCRIPTION OF EXAMPLES

### Overview

A PFA sequence typically includes a series of biphasic pulses that are short in duration and high in voltage. Each pulse has a width that may be in the order of magnitude of microseconds. The pulses are grouped together in a plurality of bursts, each burst may have a width in the order of magnitude of milliseconds. The bursts are typically separated by intra-burst delays. The intra-burst delays provide periodic cool down periods with no energy delivery to help regulate the temperature elevation associated with the energy provided by the bursts. The intra-burst delays typically have a duration in the order of magnitude of a second. As such, the time required to form a lesion with the PFA sequence is pre-dominantly defined by the number of intra-pulse delays included as well as the length of each intra-pulse delay.

Irrigation accompanying delivery of the PFA sequence also contributes to the time required to form a lesion. For each lesion created the irrigation pump ramps-up its flowrate from idle mode to active mode. The PFA sequence generator is idle for at least a portion of the ramp-up time. The ramp-up is also in the order of magnitude of a second and thereby imposes a delay. Additionally, after delivery of each PFA sequence, irrigation is maintained for a post ablation period to provide additional cooling to the lesion site as well as to clear the lesions site from the presence of blood. The post ablation period is also in the order of magnitude of a second. A physician is required to stabilize the catheter at a lesion site from the onset of irrigation ramp up to the end of the ablation. Most of this time is attributed to the accompanied delays associated with the intra-burst delays and irrigation that are needed to regulate the temperature. All these accompanying delays requires the physician to maintain catheter stability for longer than is preferred and jeopardizes efficacy if stability is not achieved.

The present disclosure describes a more efficient system and workflow for irrigating during PFA. The system and workflow support forming a lesion with a significantly shorter PFA sequence. For procedures that include forming a plurality of lesions, the workflow may reduce the number of times the flowrate of the pump is ramped-up and may reduce the delay required before forming subsequent lesion in a different site. The time the physician is required to stabilize the catheter at each lesion site may be significantly reduced without compromising the quality of the lesion and without incurring thermal damage to the tissue.

The shorter durations are afforded by reducing or eliminating the intra-burst delays, increasing the pulse widths to drive improved efficiency and reducing the overall number of pulses delivered. The workflow for irrigation supports the shorter duration PFA sequences.

The irrigation workflow is configured to maintain the active mode of irrigation for the entire duration of the PFA sequence as well as an additional preset time after the PFA sequence is delivered. During the additional preset time that the active mode is maintained, the system allows the physician to deliver additional PFA sequences to form more lesions. The additional preset time is defined to maintain the active mode for an extended period that will enable the physician to perform multiple lesions. The preset time may be between 15 seconds and 20 seconds. Maintaining the active mode on between delivery of additional PFA sequences improves temperature regulation provided with the irrigation. Once the preset time lapses, the system reverts to irrigating in an idle mode. The additional preset time is reset after each delivery of the PFA sequence. Since the PFA sequences used are significantly shortened and there is no imposed pump ramp up delay between delivery of the PFA sequence to form additional lesions, the overall duration that the flowrate is maintained in an active mode is not expected to be greater as compared to the irrigation provided with longer PFA sequences that include multiple intra-burst delays as well as pre and post ablation active mode time periods described herein.

### System/Method Description

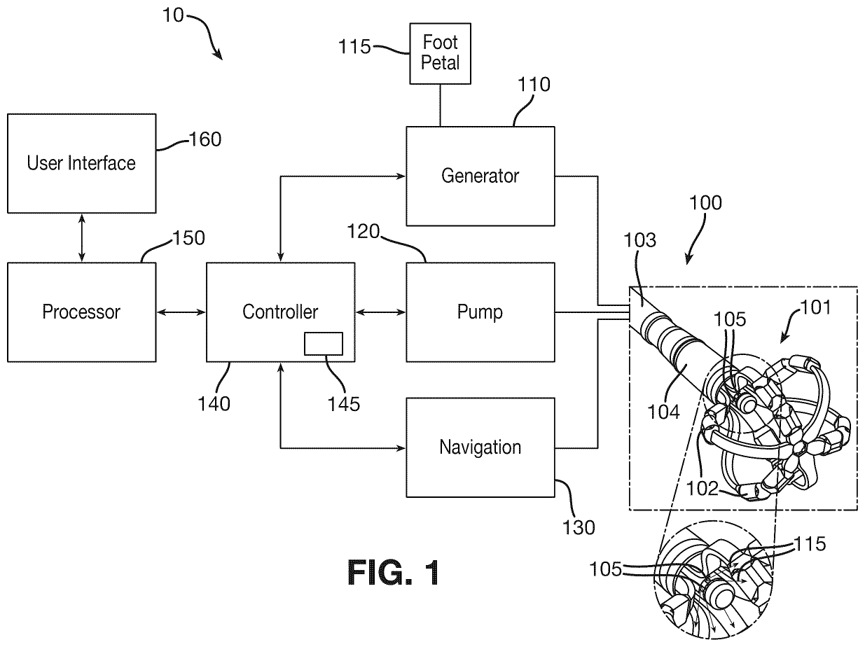

FIG. 1 a simplified block diagram of a catheter-based electrophysiology mapping and ablation system. A catheter 100 includes a distal end assembly 101 mounted at a distal end of a shaft 103. Distal end assembly includes one or more electrodes 102 for delivering PFA energy.

Electrodes 102 may also be configured to capture electrical signals from the inner wall of a heart chamber for mapping EP activity. Optionally, distal end assembly includes electrodes dedicated for mapping EP activity and electrodes dedicated to delivering PFA energy.

An irrigation tube running along shaft 103 directs irrigation toward distal end assembly 101 and expels the irrigation fluid 115 through irrigation holes 105 at an angle of approximately 45° with respect to the longitudinal axis of the shaft. In other examples, irrigation is delivered through a hollow within electrodes 102 and is expelled through holes formed on electrodes 102.

One or more position sensors 104 that are embedded in shaft 103 track location and orientation of distal end assembly 101. Optionally, position sensors 104 include one more electromagnetic coils that tracks position using a navigation system 130, e.g., a magnetic based navigation system.

Generator 110 generates PFA energy in a defined PFA sequence and delivers it to one or more of electrodes 102. Generator 110 may operate in a bipolar mode by generating a pulsed electric field between pairs of electrodes 102. Optionally, 110 may operate in a monopolar mode by generating the pulsed electric field between one or more electrodes 102 and a return electrode mounted on a patient's skin, or a return electrode mounted on shaft 103. Optionally, generator 110 enables selectively ablating with both PFA and radio frequency (RF) ablation. Generator 110 is activated by a physician, e.g., with a foot pedal 115.

Pump 120 delivers irrigation through holes 105 with a controlled flowrate. In some examples, a relatively low flowrate is used for an idle mode while catheter 100 is positioned within the heart chamber and is not ablating, e.g., during mapping and/or while the catheter is being maneuvered to reach a selected location. The flow rate for idle mode may be 1 mL/min-5 mL/min, e.g., 4 mL/min. During ablation, the flowrate is ramped up to an active mode. During an active mode, the flow rate may be 30 mL/min-50 mL/min, e.g., 40 mL/min.

A controller 140 controls operation of pump 120 by toggling pump 120 between idle mode and active mode. Toggling is in coordination with the operation of generator 110 and based on preset parameters defined by a user with user interface 160 and processor 150. User interface 160 includes one or more user interface devices for operating catheter-based electrophysiology mapping and ablation system 10. Controller 140 includes a timer and/or timing capability that is defined to reset at each ablation event. The duration of the timer is defined by the physician via user interface 160.

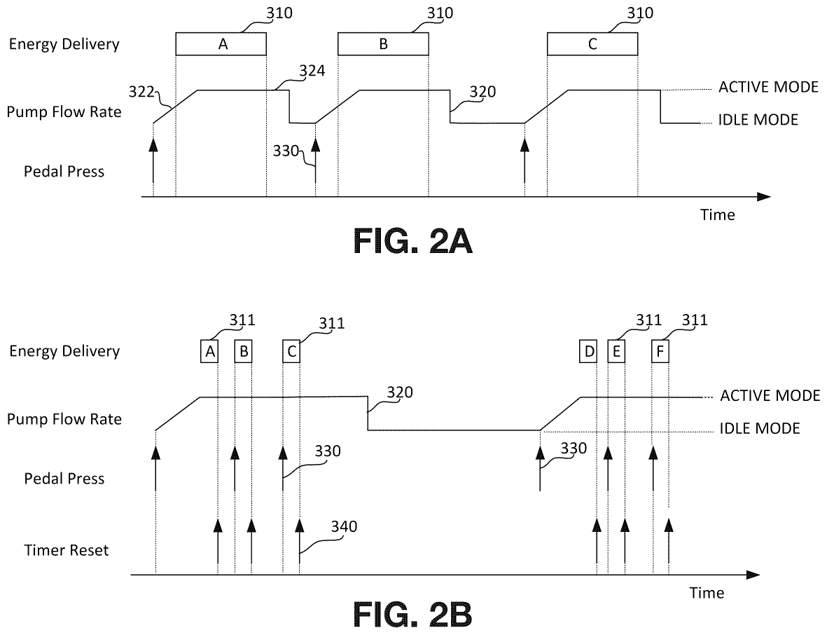

FIGS. 2A and 2B are two example timelines for activating irrigation during an electrophysiology procedure that includes Pulsed Field Ablation (PFA).

FIG. 2A shows an example workflow to form lesions in three different sites, site A, site B and site C with PFA energy. A pre-defined PFA energy sequence is delivered over each ablation duration 310. The pre-defined signal includes a plurality of bursts of pulses separated by intra-burst delays. The intra-burst delays are in the order of magnitude of seconds, e.g., 0.5 seconds to 2 seconds. Delivery of the bursts are typically accompanied by a rise in temperature. The intra-burst delay is a cooling period that enables regulating the temperature over ablation duration 310 to maintain the temperature below a desired threshold, e.g., 50° C.

Each bursts includes a series of bi-phasic pulses having a defined pulse width, pulse amplitude and optionally an inter-pulse delay and/or an inter-phase delay. The burst parameters are defined to provide a desired lesion with non-thermal PFA. Duration of each of the plurality of bursts is in the order of magnitude of milliseconds.

Ablation is initiated by a physician pressing foot pedal 115. Controller 140 (and generator 110) identifies pedal press event 330 and in response toggles activity of pump 120 from idle mode, e.g., 4 mL/min to active mode, e.g., 40 ml/min. Pump 120 ramps its flowrate over a ramp-up period 322. The ramp-up period needed is pre-determined.

Controller 140 concurrently halts delivery of PFA ablation delivery until pump 120 reaches at least 60%-80% of the active mode flowrate. The ramp-up period may be 3-5 seconds and controller 140 may initiate delivery of the PFA energy after 2-4 seconds. The delay ensures that the available irrigation is sufficient to counterbalance rise in temperature due to the delivery of PFA energy.

Controller 140 maintains operation of pump 120 in active mode for a predefined post-ablation period 324 that is pre-defined. The pre-defined post-ablation period 324 lasts 2 seconds to 7 seconds, e.g., 5 seconds. During post-ablation period 324, controller 140 blocks any additional delivery of PFA energy. Irrigation during post-ablation period 324 washes away blood from the vicinity of the ablation site to avoid intravascular hemolysis and also provides additional cooling to the lesion site and the electrodes 102. At the termination of post-ablation period 324, controller 140 toggles pump 120 to idle mode.

A physician is required to maintain catheter 100 at a stabilized location over the ramp-up period and until pump 120 is toggled back to idle mode for each lesion created. The overall period at which the physician would be required to maintain catheter 100 at a stabilized location based on the workflow in FIG. 2A may be between 10-20 seconds. When considering that the total duration at PFA energy is delivered only about 1 second, the need for the physician to maintain catheter 100 at a stabilized location for a duration of 10-20 seconds may be perceived as excessive and may feel burdensome. The rest of the 10-20 seconds accommodates the intra-burst delays as well as the ramp-up flow period and the post ablation irrigation period.

FIG. 2B shows another example workflow with PFA that provides an expediated alternative to the workflow shown in FIG. 2A. The workflow in FIG. 2B applies an alternate PFA sequence that has an ablation duration 311. Ablation duration 311 is significantly shorter than ablation duration 310 without compromising quality of the formed lesion. The reduction in duration is based on reducing the number of intra-burst delays and reducing the overall number of biphasic pulses delivered. In some examples, the alternate PFA sequence does not include any intra-burst delays. The lesion quality is maintained by increase the pulse widths relative to those used over duration 310 and modifying the method for operating pump 120. Amplitude of the biphasic pulses delivered over ablation duration 310 and ablation duration 311 may be the same.

Wider pulses are typically more efficient than narrower pulses and therefore less energy and fewer pulses are needed to form a desired lesion. Although, ablating with more efficient pulses reduces the overall energy delivered, the wider pulse widths are typically accompanied by a sharper temperature elevation.

The system and method of irrigation as described herein addresses the challenge of regulating the temperature elevation associated with increasing pulse width and the temperature elevation associated with reducing or eliminating the intra-burst delays and enables expediating the ablation procedure.

In the workflow depicted in FIG. 2B, for the first lesion formed, controller 140 delays delivery of the PFA sequence until pump 120 is fully ramped-up to active mode. However, once pump 120 is ramped-up to active mode the active mode is maintained and more than one lesion may be formed, e.g., generator 110 may deliver energy to form lesions at site A, site B, and site C while pump 120 is continuously maintained in active mode.

Every pedal press event 330 initiates a reset of the timer 145. As long as timer 145 is ON, the active mode for ablation is maintained and additional lesions may be formed without a wait period. Every additional pedal press event 330 resets timer 145. If timer 145 turns OFF, controller 140 toggles pump 120 to idle mode and new ramp-up period would be needed if physician would like to form additional lesions. Reset may occur at the termination of ablation duration 311 as shown in FIG. 2B or for example at the onset of ablation duration 311. Duration of timer 145 is preset by the physician.

For example, if a physician pauses the ablation process and operates catheter 100 to collect intra-cardiac electrograms, controller 140 would toggle to idle mode at the end of the preset time, e.g., when timer 145 turns OFF. At the first pedal press event 330 as well subsequent pedal press events that occur while the pump is in an idle mode, controller 140 will block delivery of the PFA energy until the pump has ramped up to the full flowrate at the ablation mode. Since the temperature elevation is expected to be sharp, waiting for the full ramp-up may help counteract the sharp rate of elevation.

Since ablation duration 311 is significantly shorter as compared to ablation duration 310, the overall time that controller 140 operates pump 120 at active mode based on the workflow in FIG. 2B is expected to be shorter as compared to that of workflow described in FIG. 2A for forming a same number and quality of lesions.

The present inventors have found that the modifications depicted in FIG. 2B can significantly reduce the time that the physician is required to maintain catheter 100 at a stabilized location during ablation as well as the overall time of an ablation procedure that includes multiple lesions without compromising the temperature regulation and lesion quality. Furthermore, the volume of irrigation fluid that is expelled may also be reduced based on the modifications depicted in FIG. 2B.

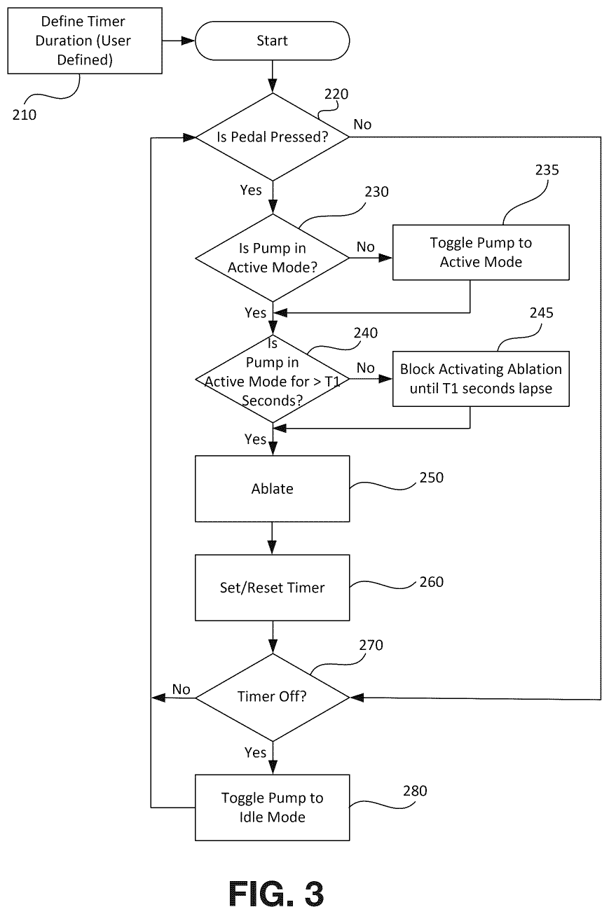

FIG. 3 is a simplified flow chart of an example method for controlling flow rate of irrigation during an electrophysiology procedure that includes PFA. In a preliminary step, a physician will select the timer duration that would be suitable via a user interface 160. For example, more experienced physicians and/or for procedures that are more standard, a physician may be able to advance from one lesion site to the next very quickly and may select to operate with a relatively short timer duration. For physician's that may be less experienced and/or for procedures that may be more complex, a longer timer duration may be desired. The timer duration defined is stored in memory associated with processor 150 and may be adjusted as needed.

At block 220, controller 140 queries generator 110 to determine if a pedal press event occurred.

At block 230 if a pedal press event is identified, controller 140 queries pump 120 to determine if pump 120 is already operating in an active mode. If not, controller toggles operation of pump 120 to active mode (block 235) and continues to block 240.

At block 240, controller determines if the active mode has been maintained for more than T1 seconds. T1 is the pre-determined ramp-up period needed by pump 120 to ramp-up from idle mode to active mode. In case T1 seconds has not lapsed, controller 140 blocks delivery of PFA energy until T1 seconds has lapsed.

When the T1 second period lapses, generator 110 immediately actuates delivery of the PFA sequence to form the lesion (block 250). Actuating ablation also triggers setting or resetting the timer (block 260).

While the timer is operating, the controller continues to query generator 110 to identify additional pedal press events (block 270). Once the timer is OFF, e.g., duration expired, controller 140 toggles operation of pump 120 to idle mode (block 280). The workflow continues starting again in block 220 to identify additional foot pedal events.

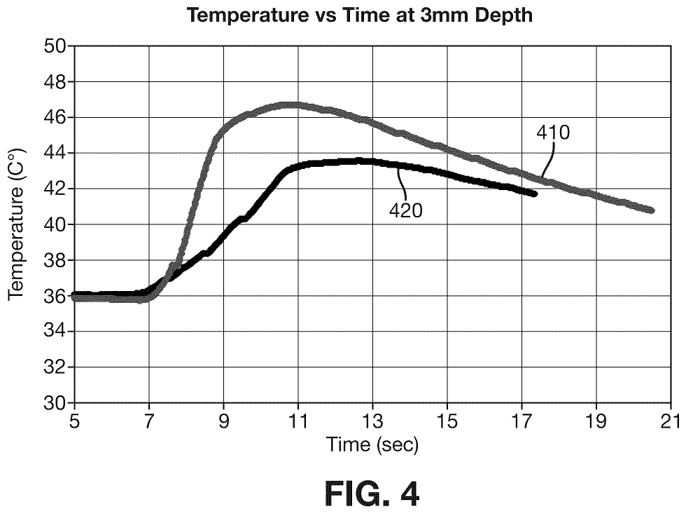

FIG. 4 shows an example temperature elevation graph showing temperature elevations for two different PFA ablation signals applied for forming a lesion at a selected site. One ablation signal has an ablation duration of 3 seconds per lesion, and another has an ablation duration of 1.2 seconds per lesion. The shorter durations are afforded by reducing or eliminating the intra-burst delays, increasing the pulse widths to drive improved efficiency and reducing the overall number of pulses delivered. Both the 3 second ablation duration and the 1.2 second ablation duration maintain a temperature below 50° C. as would be desired for PFA. The 3 second ablation duration includes intra-burst delays while the 1.2 second ablation duration does not have any intra-burst delays and thereby has a shorter ablation duration. The 1.2 second ablation duration can be seen to have a sharper temperature elevation profile as compared to the 3 second ablation duration. However, both PFA ablation signals are maintained below 50° C. and are therefore suitable for achieving non-thermal PFA ablation.

### EXAMPLES

Example 1: A method for irrigation during pulsed field ablation (PFA), the method comprising: operating a pump configured for delivering irrigation to a lesion site in idle mode; receiving a first user command to initiate delivery of a PFA sequence; toggling the pump operation from idle mode to active mode based on receiving the first user command; delivering a first PFA sequence after a pre-defined ramp-up delay based on the toggling; activating a timer, wherein the timer is preset to be ON for a defined working duration; receiving a second user command to initiate delivery of a PFA sequence; delivering a second PFA sequence with no imposed delay based on the second user command as long as the timer is ON; resetting the timer based on the second user command; and toggling the pump operation to idle mode after the defined working duration lapses.

Example 2: The method of example 1, further comprising: receiving a third user command to initiate delivery of a PFA sequence; identifying operational mode of the pump; toggling the pump operation from idle mode to active mode and delivering a third PFA sequence after a pre-defined ramp-up delay based on receiving the third user command and identifying that the pump is operating in idle mode; alternatively delivering the third PFA sequence immediately based on receiving the third user command and identifying that the pump is operating in active mode; and resetting the timer based on the third user command.

Example 3: The method of example 1 or example 2, wherein the defined working duration is selected by the user.

Example 4: The method of any one of examples 1-3, wherein the defined working duration is at least three times longer than a duration of the PFA sequence.

Example 5: The method of any one of examples 1-4, wherein the defined working duration is at least ten times longer than a duration of the PFA sequence.

Example 6: The method of any one of examples 1-5, wherein the defined working duration is configured for enabling the user to deliver multiple PFA sequences for forming multiple lesions while maintaining the pump operation in the active mode.

Example 7: The method of any one of examples 1-6, wherein each of the first and PFA sequence and second PFA sequence is defined to form a lesions in intra-cardiac tissue.

Example 8: A system for pulsed field ablation (PFA) comprising: a catheter comprising one or more electrodes configured for delivering a PFA sequence to a selected lesion site; a generator configured for delivering PFA sequences to the one or more electrodes, each sequence configured for forming a lesion in the selected lesion site; a pump configured for delivering irrigation to the lesion site, wherein the pump is configured for operating in on of an idle mode and an active mode; a controller configured for controlling operation the of pump in coordination with operation of the generator, wherein the controller includes a timer configured to be activated for a defined working duration based on the generator delivering a PFA sequence and wherein the controller is configured to: operate the pump in idle mode at startup; identify a first user command to the generator to initiate delivery of the PFA sequence; toggle the pump operation from idle mode to active mode based on identifying the first user command; delay delivery of the first PFA sequence for a pre-defined ramp-up period based on the toggling; activate the timer based on delivering the first PFA sequence; identify a second user command to the generator to initiate delivery of a PFA sequence; enable delivery of a second PFA sequence with no imposed delay based on the second user command as long as the timer is ON; reset the timer based on identifying the second user command; and toggle the pump operation to idle mode after the defined working duration lapses.

Example 9: The system of any one of example 8, wherein the defined working duration is selected by the user and stored in memory associated with the controller.

Example 10: The system of any one of example 8 or example 9, wherein the defined working duration is at least three times longer than a duration of the PFA sequence.

Example 11: The system of any one of examples 8-10, wherein the defined working duration is at least five times longer than a duration of the PFA sequence.

Example 12: The system of any one of examples 8-11, wherein the defined working duration is configured for enabling the user to deliver multiple PFA sequences for forming multiple lesions while maintaining the pump operation in the active mode.

Example 13: The system of any one of example 8-12, wherein each of the first and PFA sequence and second PFA sequence is defined to form a lesion in intra-cardiac tissue.

Example 14: The system of example 8, wherein the catheter includes at least a pair of electrodes and wherein the generator is configured for delivering the PFA sequence across the pair of the electrodes.

1. A method for irrigation during pulsed field ablation (PFA), the method comprising:
operating a pump configured for delivering irrigation to a lesion site in idle mode;
receiving a first user command to initiate delivery of a PFA sequence;
toggling the pump operation from idle mode to active mode based on receiving the first user command;
delivering a first PFA sequence after a pre-defined ramp-up delay based on the toggling;
activating a timer, wherein the timer is preset to be ON for a defined working duration;
receiving a second user command to initiate delivery of a PFA sequence;
delivering a second PFA sequence with no imposed delay based on the second user command as long as the timer is ON;
resetting the timer based on the second user command; and
toggling the pump operation to idle mode after the defined working duration lapses.

2. The method of claim 1, further comprising:
receiving a third user command to initiate delivery of a PFA sequence;
identifying operational mode of the pump;
toggling the pump operation from idle mode to active mode and delivering a third PFA sequence after a pre-defined ramp-up delay based on receiving the third user command and identifying that the pump is operating in idle mode;
alternatively delivering the third PFA sequence immediately based on receiving the third user command and identifying that the pump is operating in active mode; and
resetting the timer based on the third user command.

3. The method of claim 1, wherein the defined working duration is selected by the user.

4. The method of claim 1, wherein the defined working duration is at least three times longer than a duration of the PFA sequence.

5. The method of claim 1, wherein the defined working duration is at least ten times longer than a duration of the PFA sequence.

6. The method of claim 1, wherein the defined working duration is configured for enabling the user to deliver multiple PFA sequences for forming multiple lesions while maintaining the pump operation in the active mode.

7. The method of claim 1, wherein each of the first and PFA sequence and second PFA sequence is defined to form a lesions in intra-cardiac tissue.

8. A system for pulsed field ablation (PFA) comprising:
a catheter comprising one or more electrodes configured for delivering a PFA sequence to a selected lesion site;
a generator configured for delivering PFA sequences to the one or more electrodes, each sequence configured for forming a lesion in the selected lesion site;
a pump configured for delivering irrigation to the lesion site, wherein the pump is configured for operating in on of an idle mode and an active mode;
a controller configured for controlling operation the of pump in coordination with operation of the generator, wherein the controller includes a timer configured to be activated for a defined working duration based on the generator delivering a PFA sequence and wherein the controller is configured to:
operate the pump in idle mode at startup;
identify a first user command to the generator to initiate delivery of the PFA sequence;
toggle the pump operation from idle mode to active mode based on identifying the first user command;
delay delivery of the first PFA sequence for a pre-defined ramp-up period based on the toggling;
activate the timer based on delivering the first PFA sequence;
identify a second user command to the generator to initiate delivery of a PFA sequence;
enable delivery of a second PFA sequence with no imposed delay based on the second user command as long as the timer is ON;
reset the timer based on identifying the second user command; and
toggle the pump operation to idle mode after the defined working duration lapses.

9. The system of claim 8, wherein the controller is further configured to:
identify a third user command to the generator to initiate delivery of a PFA sequence;
identify an operational mode of the pump;
toggle the pump operation from idle mode to active mode and delay delivery of the PFA sequency by the pre-defined ramp-up delay based on receiving the third user command and identifying that the pump is operating in idle mode;
alternatively enable delivery of a second PFA sequence with no imposed delay based on receiving the third user command and identifying that the pump is operating in active mode; and
resetting the timer based on identifying the third user command.

10. The system of claim 8, wherein the defined working duration is selected by the user and stored in memory associated with the controller.

11. The system of claim 8, wherein the defined working duration is at least three times longer than a duration of the PFA sequence.

12. The system of claim 8, wherein the defined working duration is at least five times longer than a duration of the PFA sequence.

13. The system of claim 8, wherein the defined working duration is configured for enabling the user to deliver multiple PFA sequences for forming multiple lesions while maintaining the pump operation in the active mode.

14. The system of claim 8, wherein each of the first and PFA sequence and second PFA sequence is defined to form a lesion in intra-cardiac tissue.

15. The system of claim 8, wherein the catheter includes at least a pair of electrodes and wherein the generator is configured for delivering the PFA sequence across the pair of the electrodes.
